(19) United States
(12) Patent Application Publication
(10) Pub. No.: US 2009/0156587 A1
(43) Pub. Date: Jun. 18, 2009

(54) Eye Medication Formulation with Antibacterial Agent

(76) Inventor: Ravi Nallakrishnan, Westmont, IL (US)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/959,418

(22) Filed: Dec. 18, 2007

Related U.S. Application Data
(60) Provisional application No. 61/013,232, filed on Dec. 12, 2007.

(51) Int. Cl. A61K 31/5383

(52) U.S. Cl. 514/230.2

(57) ABSTRACT

A formulation for administration to the eye has at least one pharmaceutical agent such as a mydriatic agent, a cycloplegic agent, an anesthetic or a non-steroidal anti-inflammation agent combined with an anti-bacterial agent and a suitable carrier. The formulation can be made for topical or intracameral administration to the eye.

### BACKGROUND OF THE INVENTION

The present invention relates to preparations used in surgical and examination procedures in connection with the eye and, more particularly, to a formulation that includes multiple ingredients, one of which is an antibacterial agent.

This application claims priority from U.S. Patent Application Ser. No. 61/013,232, filed Dec. 12, 2007, which is incorporated herein by reference.

Dilation of the pupil of the eye is a common ophthalmic technique useful in examining the eye or in performing surgery upon the eye. Pupillary dilation gives the surgeon a greater field of vision during surgical procedures.

Dilation is carried out by the use of both mydriatic and cycloplegic agents, both of which cause the sphincter of the iris to open. In addition, cycloplegic agents also may temporarily paralyze the lens of the eye, accompanied by loss of accommodation.

In order to be effective, both such agents must penetrate the layers of the cornea, in particular the corneal epithelium order to reach the iris sphincter and the ciliary body. For topical procedures, penetration is made possible and more effective the longer the preparation remains in contact with the cornea. Where penetration is done intracamerally the agents are injected through the cornea directly into the anterior chamber.

As described in the prior art, most particularly, U.S. Patent Application Publication US2004/0013729 (Buono) a number of advantages are gained if the topically-applied agents to be used in an eye examination or in ophthalmic surgery are applied simultaneously rather than successively. For topical applications, one advantage is that successive applications of liquids containing one or more of the needed agents will not rinse or wash away any agents that have already been infused into the eye.

For intracameral applications, there is no necessity for multiple injections for individual agents if all are included in a single injection.

Another advantage to delivering multiple agents in a single operation is that no large inventory of bottles of individual drugs must be purchased, maintained, tracked and timely used.

One way to make the topical penetration of the selected agents more effective is to combine them in a carrier with a relatively high viscosity. This allows the agents and carrier to spread uniformly across the surface of the cornea and remain in place for a longer period of time than would be possible if the agents are applied separately or in a less viscous carrier.

Another frequently used agent is an anesthetic which will block any pain or other sensations caused by the use of instruments on the eye and make it possible for the patient to tolerate the examination procedure or surgical procedure. An example of an anesthetic disposed in a high-viscosity carrier is a product manufactured by CYNACON/OCuSOFT, Inc. of Rosenberg, Tex. under the name TetraVisc™, which combines a high-viscosity carrier with tetracaine hydrochloride.

Yet another frequently used agent is a non-steroidal anti-inflammatory agent (NSAIA) used to minimize or control the swelling of tissue.

The use of these agents such as these and their combination into a single topically applied composition is well described in the foregoing Buono reference. Those descriptions are incorporated herein by reference.

A frequently-incurred problem is the presence of bacterial infections after surgery. While there are a number of effective and widely used agents available for treating such infections, I have determined that there is a benefit to be realized by including an antibacterial agent together with agents topically or intracamerally applied prior to surgery. The advantages of using an antibacterial as a preventive for infection and its inclusion into a single topical formulation along with other desired or required agents makes it possible to achieve maximum penetration through the cornea by avoiding successive applications of individual agents. The advantages to using an anti-bacterial agent in an intracamerally-applied combination include avoiding the necessity for a further injection or a topical application after an intracameral injection.

### BRIEF SUMMARY OF THE INVENTION

Described generally, the invention is a composition or formulation for both topical and intracameral use in the eye which includes a combination of agents selected for the particular patient or procedure to be undertaken. For a topical preparation, these agents include one or more of a mydriatic agent, a cycloplegic agent, a high-viscosity polymer used as a carrier and an NASIA, together with an antibacterial agent and a sufficient amount of water to give the formulation its desired consistency. For an intracameral procedure the active agents would be combined in a single injection in a pharmaceutically suitable carrier that can contain water and a high-viscosity polymer.

More particularly, a first topical or intracameral formulation would include at least one of the following:

a pharmaceutically effective amount of a mydriatic agent;

a pharmaceutically effective amount of a cycloplegic agent; and

a pharmaceutically effective amount of an NASIA.

The selected agent or agents would then be combined with an antibacterial agent, a viscoelastic polymer carrier and a sufficient amount of water to give the final preparation its desired flow characteristics.

The term “pharmaceutically effective amount” as used with respect to a mydriatic agent means a sufficient amount of such an agent to cause mydriasis.

A pharmaceutically effective amount of a cycloplegic agent will be considered to be an amount sufficient to cause cycloplegia.

A pharmaceutically effective amount of an NSAIA means an amount effective to reduce or prevent unwanted swelling.

A pharmaceutically effective amount of an antibacterial agent means an amount of such an agent sufficient to prevent or treat a bacterial infection.

A pharmaceutically effective amount of an anesthetic means an amount of a selected anesthetic sufficient to reduce or eliminate patient discomfort during the procedure or examination.

Examples of suitable mydriatic agents are phenylephrine, naphazoline, or epinephrine. The use of other mydriatic agents is also anticipated.

Examples of suitable cycloplegic agents are tropicamide, cyclopentioate, scopolamine, homotropine and atropine. The use of other cycloplegic agents is also anticipated

Examples of suitable NSAIA agents are propylmethylcelluose, methylcellulose, carboxymethylcellulose, hyaluronate and chondroitin sulfate. The use of other NSAIA agents is also anticipated

Suitable anesthetics include many agents, examples of which are lidocaine, pilocarpine, tetracaine, proparacaine and bupivicaine. The use of other anesthetic agents is also anticipated.

The viscoelastic polymer used in connection with the present invention is available in a number of commercial products, a number of which used hydroxypropylmethylcellulose (HPMC). The amount of viscoelastic polymer is typically adjusted to meet the needs of the surgeon in connection and dictated by the agents in the particular formulation and the use to which they will be put.

I have determined that a number of antibacterial agents are suitable for use with the present invention. Such agents are typically identified as quinolones, a family of broad spectrum antibiotics and, in particular, fluoroquinolones which have become a community standard for use in connection with cataract surgery.

Three such antibacterial agents are moxifloxacin, sold under the Trademark VIGAMOX by Alcon Laboratories of Forth Worth, Tex., the agent gatifloxatin sold under the Trademark ZYMAR by Allergan, Inc. of Irvine, Calif., and levofloxatin sold under the trademark QUIXIN by Santen. Oy, Tampere, Finland.

For intracameral use, a desired combination including pharmaceutically effective amounts of one or more of a cycloplegic agent, a mydriatic agent or an NSAIA is combined with an antibacterial agent and a carrier suitable for intracameral injection.

### DETAILED DESCRIPTION OF THE INVENTION

The following examples will demonstrate the nature of the present invention with respect to topical preparations.

### Example 1

A pharmaceutically effective amount of a mydriatic agent is combined with a pharmaceutically effective amount of an anesthetic and a pharmaceutically effective amount of an antibacterial agent in a viscoelastic carrier and is then diluted with water to a desired final viscosity.

### Example 2

A pharmaceutically effective amount of a mydriatric agent is combined with a pharmaceutically effective amount of a cycloplegic agent and a pharmaceutically effective amount of an antibacterial agent in a viscoelastic carrier and is then diluted with water to a desired final viscosity.

### Example 3

A pharmaceutically effective amount of a mydriatric agent is combined with a pharmaceutically effective amount of a cycloplegic agent, a pharmaceutically effective amount of an anesthetic and a pharmaceutically effective amount of an antibacterial agent in a viscoelastic carrier and is then diluted with water to a desired final viscosity.

### Example 4

A pharmaceutically effective amount of an anesthetic and a pharmaceutically effective amount of an antibacterial agent are combined in a viscoelastic carrier and then diluted with water to a desired final viscosity.

### Example 5

A pharmaceutically effective amount of an anesthetic, a pharmaceutically effective amount of a non-steroidal anti-inflammatory agent and a pharmaceutically effective amount of an antibacterial agent are combined in a viscoelastic carrier and then diluted with water to a desired final viscosity.

### Example 6

A pharmaceutically effective amount of a mydriatric agent is combined with a pharmaceutically effective amount of a cycloplegic agent, a pharmaceutically effective amount of an anesthetic, a pharmaceutically effective amount of a non-steroidal anti-inflammatory agent and a pharmaceutically effective amount of an antibacterial agent in a viscoelastic carrier and then diluted with water to a desired final viscosity.

The following examples will demonstrate the nature of the present invention with respect to intracameral preparations.

### Example 7

A pharmaceutically effective amount of a mydriatic agent is combined with a pharmaceutically effective amount of an anesthetic and a pharmaceutically effective amount of an antibacterial agent in a carrier suitable for intracameral injection.

### Example 8

A pharmaceutically effective amount of a mydriatric agent is combined with a pharmaceutically effective amount of a cycloplegic agent and a pharmaceutically effective amount of an antibacterial agent in a carrier suitable for intracameral injection.

### Example 9

A pharmaceutically effective amount of a mydriatric agent is combined with a pharmaceutically effective amount of a cycloplegic agent, a pharmaceutically effective amount of an anesthetic and a pharmaceutically effective amount of an antibacterial agent in a carrier suitable for intracameral injection.

### Example 10

A pharmaceutically effective amount of an anesthetic and a pharmaceutically effective amount of an antibacterial agent are combined in a carrier suitable for intracameral injection.

### Example 11

A pharmaceutically effective amount of an anesthetic, a pharmaceutically effective amount of a non-steroidal anti-inflammatory agent and a pharmaceutically effective amount of an antibacterial agent are combined in a carrier suitable for intracameral injection.

### Example 12

A pharmaceutically effective amount of a mydriatric agent is combined with a pharmaceutically effective amount of a cycloplegic agent, a pharmaceutically effective amount of an anesthetic, a pharmaceutically effective amount of a non-steroidal anti-inflammatory agent and a pharmaceutically effective amount of an antibacterial agent in a carrier suitable for intracameral injection.

While the foregoing describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

I claim:

1. A composition for administration to the eye, said composition comprising:
at least one pharmaceutical agent in an amount to produce a desired pharmaceutical result;
an antibacterial agent present in a pharmaceutically effective amount to treat or prevent bacterial infection; and
a physiologically acceptable carrier for said pharmaceutical and antibacterial agents.

2. The apparatus as recited in claim 1 wherein said pharmaceutical agent is present in a pharmaceutically effective amount and is chosen from the group of a mydriatic agent, a cycloplegic agent, an anesthetic agent or a non-steroidal anti-inflammation agent.

3. The apparatus as recited in claim 1 wherein said composition further comprises pharmaceutically effective amounts of a mydriatic agent, a cycloplegic agent, and an anesthetic agent.

4. The apparatus as recited in claim 1 wherein said composition further comprises pharmaceutically effective amounts of a mydriatic agent and an anesthetic agent.

5. The apparatus as recited in claim 1 wherein said composition further comprises pharmaceutically effective amounts of a cycloplegic agent, and an anesthetic agent.

6. The apparatus as recited in claim 1 wherein said at least one pharmaceutical agent, said carrier and said anti-bacterial agent are suitable for topical administration to said eye.

7. The apparatus as recited in claim 1 wherein said at least one pharmaceutical agent, said carrier and said anti-bacterial agent are suitable for intracameral injection into said eye.
